(19) United States
(12) Patent Application Publication
(10) Pub. No.: US 2015/0017131 A1
(43) Pub. Date: Jan. 15, 2015

(54) HAIR FOLLICLES MADE EX VIVO THAT CAN BE INSERTED INTO A RECIPIENT FOR HAIR RESTORATION

(71) Applicant: Jordan FABRIKANT, El Cajon, CA (US)

(72) Inventor: Jordan FABRIKANT, El Cajon, CA (US)

(21) Appl. No.: 14/308,798

(22) Filed: Jun. 19, 2014

Related U.S. Application Data
(60) Provisional application No. 61/843,922, filed on Jul. 9, 2013.

(51) Int. Cl. C12N 5/071; C12N 5/00; B29C 67/00; A61K 35/36

(52) U.S. Cl. CPC C12N 5/0627; A61K 35/36; C12N 5/0062; B29C 67/0059; C12N 2506/094; C12N 2501/10; C12N 2502/09; B29K 2089/00; B29K 2995/0056
USPC 424/93.7; 435/377; 264/308

(57) ABSTRACT

The present invention recognizes that there exists a long felt need for reliable hair growth methods and compositions that do not suffer from side effects and limitations of current technologies, such as surgery using a subject's own hair and pharmaceutical compositions. A first aspect of the present invention is a method of making at least one three dimensional collection of cells capable of forming a functional hair follicle. A second aspect of the present invention is a product produced by the method of making at least one three dimensional collection of cells capable of forming a functional hair follicle of the present invention. A third aspect of the present invention is a method of making at least one functional hair follicle. A fourth aspect of the present invention is a product produced by the method of making at least one functional hair follicle of the present invention. A fifth aspect of the present invention is a method of hair growth in a subject using at least one three dimensional collection of cells capable of forming a functional hair follicle of the present invention. A sixth aspect of the present invention is a method of hair growth in a subject using at least one functional hair follicle of the present invention.

The present application claims benefit of priority to U.S. Provisional Application Ser. No. 61/843,922, filed Jul. 9, 2013, entitled “Hair Follicles Made Ex Vivo That Can Be Inserted Into a Recipient For Hair Restoration,” which is herein incorporated by reference in its entirety.

### TECHNICAL FIELD

The present invention generally relates generally to the fields of hair growth utilizing cells, such as but not limited to stem cells, fully differentiated cells, or cells anywhere in between these two benchmarks, and ex vivo created and replicated hair follicles, and preferably printing technologies to create biologically representative hair follicles that are ready for insertion into a subject such as a human, at the proper angle with correct spatial orientation, by a hair restoration surgeon or other health care provider using traditional methods. These ex vivo created and replicated hair follicles may be inserted in single, double, triple, or multiple follicular units into a subject, such as in the scalp, to increase the aesthetic outcome of a hair restoration procedures.

### BACKGROUND

There exists a long felt need for reliable and effective hair growth methods and compositions that do not suffer from side effects and limitations of current technologies, such as hair restoration surgery using a subject's own hair, pharmaceutical compositions, cells, and tissue engineering.

US Published Patent Application No. 2010/0034787 to Naughton (incorporated herein by reference in its entirety) provides a summary of hair follicle structure, cell types, and their origin, particularly at column 1. See particularly paragraphs [0002], [0003] and [0004], which are provided in whole or in part below.

The hair follicles of mammals develop from extensions of the embryonic epidermis that differentiate into three different layers of the mature hair. The central layer forms the hair shaft while the outer most layer forms the outer root sheath. The middle cylinder forms the inner root sheath that guides the hair shaft outward from the epidermis. At the base of the hair follicle is the dermal papilla, a pear shaped structure formed by a group of fibroblast cells derived from the mesoderm. The dermal papilla directs the embryonic generation of the hair follicle and also controls the regeneration of the hair follicle throughout its lifecycle. Thickness of the hair fiber correlates with the size of the dermal papilla. A basement membrane or basement lamina demarcates the dermal papilla cells from the hair fiber/sheath cells. US Published Patent Application No. 2010/0034787 to Naughton at paragraph [0002].

Normal mature hair follicles undergo a regenerative cycle defined by a growth stage (anagen), a degenerative stage (catagen), a resting stage (telogen), and a shedding stage (exogen). Anagen is the phase of hair follicle growth extending from the telogen stage to the beginning of the catagen stage and involves regrowth of the cycling part of the hair follicle. In anagen, dermal papilla fibroblasts secrete numerous growth factors that maintain active proliferation and differentiation of keratinocytes of the proximal hair bulb that forms the hair fiber. The length of the anagen phase, which may be further subdivided into six subphases (Anagen I-VI), is limited and is determinative of the hair shaft length. A longer anagen phase produces longer hair fibers. Anagen may be initiated in some instances by wounding of the hair follicle by plucking, vigorous shaving, or chemical insult (e.g., depilatory agents). US Published Patent Application No. 2010/0034787 to Naughton at paragraph [0003].

After the anagen stage, follicle growth stops and is followed by the catagen stage, at which time the fibroblasts retract from the basement membrane and the size of the papilla decreases. A decline in secretion of growth factors by the dermal papilla results in the reduction of proliferation and differentiation of hair matrix keratinocytes, resulting in cessation of hair shaft production. Epithelial cell death is prominent within the regressing follicle. At the end of catagen, follicular elements are lost around the papilla fibroblasts. As the hair follicle transitions to the telogen stage, the remaining hair takes on a club-shaped appearance with a small bud of the epithelial column being present at the follicle base. The telogen follicle rests in the dermis above the group of papilla fibroblasts. There are no further changes in the hair follicle until re-initiation of anagen. US Published Patent Application No. 2010/0034787 to Naughton at paragraph [0004].

Hair transplant surgery has been used for cosmetic purposes for hair loss for quite some time. The problem with hair transplantation is that there are only a finite number of hairs that can be extracted from a donor region, an area of the scalp that the patient grows hair that does not go bald or thin. A patient may run out of donor hairs, which may result in an insufficient supply to cover the necessary bald area that needs transplanted hair follicles. One way to avoid insufficient supply of donor hair would be to create/grow donor hair follicles in a laboratory that can be later inserted into the scalp of a bald person and continue growing, which is one aspect of the present invention.

Another problem with hair transplants is the donor hair has to be extracted from the donor region of the scalp. The process leaves a linear scar when the strip method is used, and this leaves multiple punctate scars on a patient's scalp when the follicular unit extraction method is used. A way to avoid making these scars would be to produce donor follicles that do not require large or multiple skin excisions from the patient's scalp, which is one aspect of the present invention.

One advantage to a hair transplant is that the harvested donor hairs are inserted into the recipient site by a physician in a planned and controlled density and in the desired direction and angle. Also, quality donor hairs are selected from surgical extraction so the physician has control in deciding the quality of the hair that will be inserted into the recipient site.

Pharmaceuticals have also been used for cosmetic purposes for hair loss for quite some time. Minoxidil, for example, has to be applied daily to have the treatment continue to work effectively. Minoxidil's efficacy and reliability is much less than a hair transplant and has to be continually applied long term to continue to maintain hair growth.

Finasteride, a pill that blocks Dihydrotestosterone by inhibiting the enzyme 5 alpha reductase, can cause side effects in men such as impotence, decreased sexual desire, and a reduced sperm count. There have also been case reports which attribute the cause of breast cancer in men to using Finasteride. In women, it can cause birth defects if taken when pregnant. Finasteride also has to be used for a long period of time and discontinuation will discontinue efficacy.

Stem cells and hair growth complex injections have also been used for cosmetic purposes for hair loss, and are undergoing clinical trials. The problem with injecting stem cells and/or hair growth complexes is that growth of hair follicles is unpredictable. The number of hairs grown is unpredictable, the quality of hair follicles is unpredictable, and the direction of hair growth is unpredictable.

As for tissue engineering and related compositions and methods for use for cosmetic purposes, U.S. Pat. No. 7,198,641 to Barrows relates to porous, bioabsorable scaffolds for tissue engineering of human hair follicles. Also, U.S. Pat. No. 7,597,885 to Barrows et al. relates to a scaffold that is constructed to mimic the architecture of a native hair follicle. In addition, U.S. Pat. No. 7,985,537 to Zheng et al., relates to methods for determining hair inductive properties of a composition by injecting dermal cells and epidermal cells into the skin of a mammal to determine whether a hair follicle can be formed. Furthermore, US Published Patent Application No. 2012/0095445 to Zheng et al. relates to methods and compositions for increasing trichogenicity of cells in culture that can utilize sonic hedge-hog pathway agonists. Also, US Published Patent Application No. 2012/0156228 to Steinberg et al. relates to methods, kits, and compositions for generating new hair follicles and growing hair on a subject. Furthermore, US Published Patent Application No. 2011/0130711 to Kedernab et al. relates to segmentation of hair follicles into small subunits to form new follicles. In addition, U.S. Pat. No. 8,252,749 to Steinberg et al. relates to methods, kits, and compositions for generating new hair follicles and growing hair on a subject. Also, US Published Patent Application No. 2010/0305699 relates to methods and compositions for anchoring natural or artificial hairs in a subject.

US Published Patent Application No. 2010/0178683 to Barrowes et al. relates to hair grafts utilizing tissue engineered skin that includes an epidermal layer, a dermal layer, hair follicle progenitor cells, a scaffold, and related methods of making and using same.

US Published Patent Application No. 2010/0034787 to Naughton relates to growth factors derived from a three dimensional tissue for administration to a subject and three dimensional tissue for administration to a subject to promote hair growth.

US Published Patent Application No. 2005/0214344 and U.S. Pat. No. 7,597,885, to Barrowes et al. relates to an improved scaffold for use in methods of hair restoration such as in Barrowes, US Published Patent Application No. 2010/0178683.

No known method has created a fully or substantially fully formed and developed terminal hair, or parts or portions thereof, or precursors thereof for implantation into a subject, optionally utilizing printing technologies, such as but not limited to, 3D printing technologies as set forth in the present invention, which are just some of the aspects and embodiments of the present invention set forth herein. As set forth above, previous methods have been focused on implanting materials or stem cells that grow new hairs from an earlier stage in hair development, and do not utilize printing technologies.

### SUMMARY

The present invention recognizes that there exists a long felt need for reliable hair growth methods and compositions that do not suffer from side effects and limitations of current technologies, such as surgery using a subject's own hair and pharmaceutical compositions.

A first aspect of the present invention is a method of making at least one three dimensional collection of cells capable of forming a functional hair follicle.

A second aspect of the present invention is a product produced by the method of making at least one three dimensional collection of cells capable of forming a functional hair follicle of the present invention.

A third aspect of the present invention is a method of making at least one functional hair follicle.

A fourth aspect of the present invention is a product produced by the method of making at least one functional hair follicle of the present invention.

A fifth aspect of the present invention is a method of hair growth in a subject using at least one three dimensional collection of cells capable of forming a functional hair follicle of the present invention.

A sixth aspect of the present invention is a method of hair growth in a subject using at least one functional hair follicle of the present invention.

### BRIEF DESCRIPTION OF THE FIGURES

FIG. 1 generally depicts a 3D bioprinter capable of producing a product of the present invention.

FIG. 2 depicts a general schematic of printing a collection of cells capable forming at least one hair follicle.

FIG. 3A generally depicts a subject whose hair follicles are being harvested as a source of cells for use in the present invention.

FIG. 3B generally depicts a tissue sample that includes a hair follicle obtained in FIG. 3A.

FIG. 4 generally depicts a subject in need or desiring a follicular transplant receiving a follicular transplant using a product of the present invention.

FIG. 5 generally depicts a collage of FIG. 1, FIG. 2, FIG. 3A, FIG. 3B, and FIG. 4 depicting one preferred aspect of the present invention.

FIG. 6 generally depicts to a 3D printer nozzle printing at least one printed cell layer.

FIG. 7 generally depicts 3D printing of a patch of follicle-rich implantable structure in a shape that generally matches the topology of the area in general on a subject that is to receive the 3D printed patch of follicle-rich skin.

### DETAILED DESCRIPTION OF THE INVENTION

### Definitions

Unless defined otherwise, all technical and scientific terms used herein have the same meaning as commonly understood by one of ordinary skill in the art to which this invention belongs. Generally, the nomenclature used herein and the laboratory procedures in cell culture, chemistry, microbiology, molecular biology, cell science, and other applicable technologies described below are well known and commonly employed in the art. Conventional methods are used for these procedures, such as those provided in the art and various general references such as Bolognia et al, Dermatology, Third Edition, Elsevier, 2008. Where a term is provided in the singular, the inventors also contemplate the plural of that term. The nomenclature used herein and the laboratory procedures described below are those well known and commonly employed in the art unless set forth otherwise. As employed throughout the disclosure, the following terms, unless otherwise indicated, shall be understood to have the following meanings:

“Subject” refers to any organism having hair follicle structures, particularly mammals. The subject can be a human or non-human primate, preferably human. Any other mammal is included, such as but not limited to veterinary, agricultural, wildlife, and test or laboratory animals. Test or laboratory animals include, but are not limited to, mice, rats, guinea pigs, dogs, cats, pigs and ferrets. A subject can be a patient, including a human patient.

Other technical terms used herein have their ordinary meaning in the art that they are used, as exemplified by a variety of technical dictionaries.

### Introduction

The present invention recognizes that there exists a long felt need for reliable hair growth methods and compositions that do not suffer from side effects and limitations of current technologies, such as surgery using a subject's own hair and pharmaceutical compositions.

As a non-limiting introduction to the breath of the present invention, the present invention includes several general and useful aspects, including:
1) A method of making at least one three dimensional collection of cells capable of forming a functional hair follicle.
2) A product produced by the method of making at least one three dimensional collection of cells capable of forming a functional hair follicle of the present invention.
3) A method of making at least one functional hair follicle.
4) A product produced by the method of making at least one functional hair follicle of the present invention.
5) A method of hair growth in a subject using at least one three dimensional collection of cells capable of forming a functional hair follicle of the present invention.
6) A method of hair growth in a subject using at least one functional hair follicle of the present invention.

These aspects of the invention, as well as others described herein, can be achieved by using the methods, articles of manufacture and compositions of matter described herein. To gain a full appreciation of the scope of the present invention, it will be further recognized that various aspects of the present invention can be combined to make desirable embodiments of the invention.

Some aspects of the present invention include the manufactured premade 3-dimensional hair follicles in the laboratory which can be implanted into a patient's bald or thinning scalp at the proper angle and orientation such as done in current hair restoration surgery and then proceed to grow naturally. This aspect of the present invention combines the advantages of hair multiplication, which reduces the need for large scale surgical extraction of donor hair follicles avoiding scars as well as creating a large/limitless supply of donor hair, with the advantages of a hair transplant, the insertion of hair follicles with adequate quality, density, angle, and direction to control and predict outcome of recipient site hair follicle growth.

Also, if a pre-molded or pre-3D printed hair follicle was manufactured and implanted into a patient's scalp, the physician could control for hair quality, number of hair follicles implanted, and the angle and direction of hair growth. The advantage to this method is that there is theoretically a large or essentially limitless supply of donor hair follicles. Previous methods that require harvesting donor hairs have too small of a follicle supply, leaving the hair restoration surgeon unable to provide the patient with new hair on parts of the scalp. In addition, there is no need for surgical extraction of large amounts of donor hair follicles on a large scale.

General Summary of Methods of Making Functional Hair Follicles Using 3D Printing and the Resulting Functional Hair Follicles

The present invention provides solutions to the problems associated with hair growth. One aspect of the present invention is to create premade hair follicles that can be surgically implanted into the recipient area of the bald or thinning scalp, i.e. the area of the scalp that has gone bald or thinned, and is in need of hair follicles. The hair follicles are viable once implanted and preferably continually grow once implanted in the scalp, in a similar way that a hair follicle that was transplanted from the donor region. These hair follicles can be grown in a lab with growth factors, growth complexes, stem cells, cell multiplication techniques, and synthetic biological materials. Once the materials are grown, duplicated, and engineered, the materials can then be assembled into a form of a hair follicle that provides the desired structure and geometry to be viable to grow hair or material representative biologically of hair.

The process of taking the material components of a hair follicle and giving it the necessary structure can be accomplished in different ways. One aspect of the present invention is the use of a 3D printer to print three-dimensional hair follicles using the appropriate biological materials such as cells, stem cells, and synthetic biological molecules as the “ink.” Another aspect of the present invention is to make and culture three-dimensional “molds” (or biological structures representative of hair follicles”) of hair follicles using materials such as cells, stem cells and synthetic biological materials. The technical, patent, and media literature is replete with methods and devices for printing cells, including stem cells, to produce tissues, structures, organs and the like. See, for example, Faulker-Jones et al., Biofabrication 5(2013)015013, and US Patent Application Publication No. US 2011/0315227, US Published Patent Application No. 2012/0224755 to Wu, U.S. Pat. No. 7,087,200 to Taboas et al, U.S. Pat. No. 8,260,589 to Kumar, Mironove et al. “Organ Printing: Computer-aided Jet-based 3D tissue engineering,” organprint.missouri.edu/www/fibr-pub/mironov03-157.pdf (public availability date not available, copy obtained Jun. 27, 2013), Marga et al. “Toward engineering functional organ modules by additive manufacturing” Biofacation, 4:1012 (2012), Khatiwala et al. “3D cell bioprinting for regenerative medicine research and therapies” Gene Therapy and Regulation 7(1):1-19 (2012), www.Organonovo.com materials relating to 3D cell printing (Jul. 5, 2012), “3D bioprinting with autologous cells could prevent organ transplant rejections, says Organovo Exec. VP” May 10, 2013, medtechinsider.com/archives/3140, Takebe et al, “Self-organization of human hepatic organoid by recapitulating organogenesis in vitro” Transplantation Proceedings 44:1018-1020 (2012), and Mannoor et al. “3D printed bionic ears” Nano Lett. 13:2634-2639 (2013), the technologies therein are applicable for use in the present invention and are incorporated by reference herein.

This type of process can be done on a small, medium or large scale to replicate in a laboratory, such as but not limited to, from between about 1,000 to about 10,000, or more or less than that range as a task at hand dictates, 3-dimensional donor hair follicles that can then be inserted into the recipient region of a patient's scalp by a physician or other appropriate medical practitioner or professional. Thus, the only surgery done during this process is recipient hair placement. Donor hair extraction would thus be essentially eliminated on a large scale of hair follicles as would be the case with traditional surgeries. Donor hair extraction of a very small amount of hair follicles, for example between about 10 and about 100, or more or less depending on the task at hand, could be needed to obtain and multiply hair cells, stem cells, growth factors, growth complexes, and biological materials that are desirable to create, grow, and mold the 3D structure of a hair follicle that will later be implanted into the recipient area.

The pre-molded or pre-3D printed hair follicles can use materials extracted from the patient that they will be implanted into. The reason for the same cells and biological materials being implanted into the same patient that they would be extracted and multiplied is to prevent an immune rejection reaction. The body recognizes and distinguishes materials from self and non-self and would not create an immune response to the implanted donor hairs grown in the lab. Thus, there would not be a rejection of the implanted hair follicles by the immune system, either graft versus host or host versus graft type rejections.

In the alternative, there would be a supply of universal donor hair follicles that are created, grown, and multiplied as explained by the above process that can be injected into any patient. This can be done by eliminating antigens and haptens from the donor patient's hair follicles so that the recipient patient's immune system would not recognize the implanted hair follicles as non-self and therefore would not reject them. Another way to create, grow, and multiply a supply of universal donor hair follicles that can be injected into any patient without rejection of the patient's immune system would be to create, grow, and multiply 3 dimensional hair follicle structures with synthetic biological materials that do not require any tissue, molecules, or hair to be extracted from a patient. There would therefore not be non-self biological materials that could be recognized by a different patient's immune system to begin with, therefore avoiding immune system rejection.

General Summary of Methods of Hair Re-Growth Using the Composition of the Present Invention

As set forth in Example II below, hair follicle structures made using the methods of the present invention are inserted into a subject at a desired location or multiple locations. Routine methods for hair transplant surgery are used.

GENERAL SUMMARY OF THE FIGURES

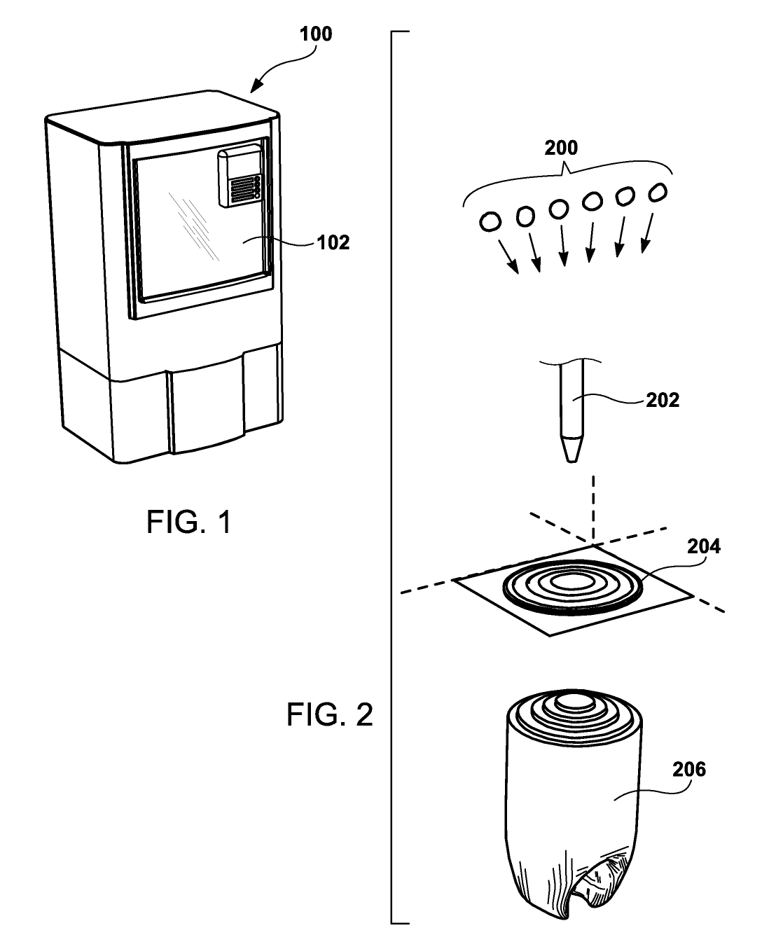

FIG. 1 depicts a 3D bioprinter (100) capable of producing a product of the present invention. The 3D bioprinter includes a sterile or substantially sterile printing chamber (102) or sterile room or substantially sterile room, which allows for maintaining of a sterile or substantially sterile environment during the 3D printing process such as for the production of a product of the present invention.

FIG. 2 is a schematic depicting cells (200) preferably inside at least one cartridge of the 3D bioprinter, at least one printer nozzle (202) printing the product of the present invention, and at least one printed cell layer (204), on an X, Y, and Z plane, printed by the 3D bioprinter through the at least one printer nozzle. The at least one printed cell layers are printed on top of one another and built up along the z-axis, to create at least one partial hair follicle (206), and eventually at least one complete or partially complete collection of cells forming at least one hair follicle or a collection of cells capable forming at least one hair follicle.

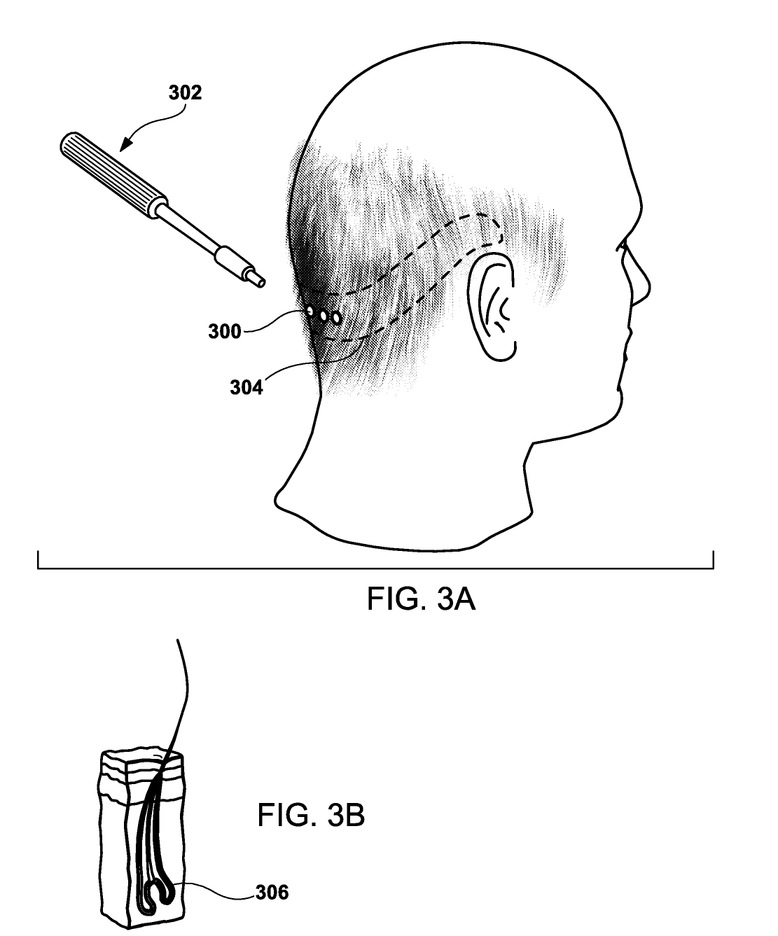

FIG. 3A generally refers to a subject whose hair follicles (300) are being harvested as a source of cells for use in the present invention using a follicle extraction tool (302), preferably from a follicle extraction zone of the subject (304).

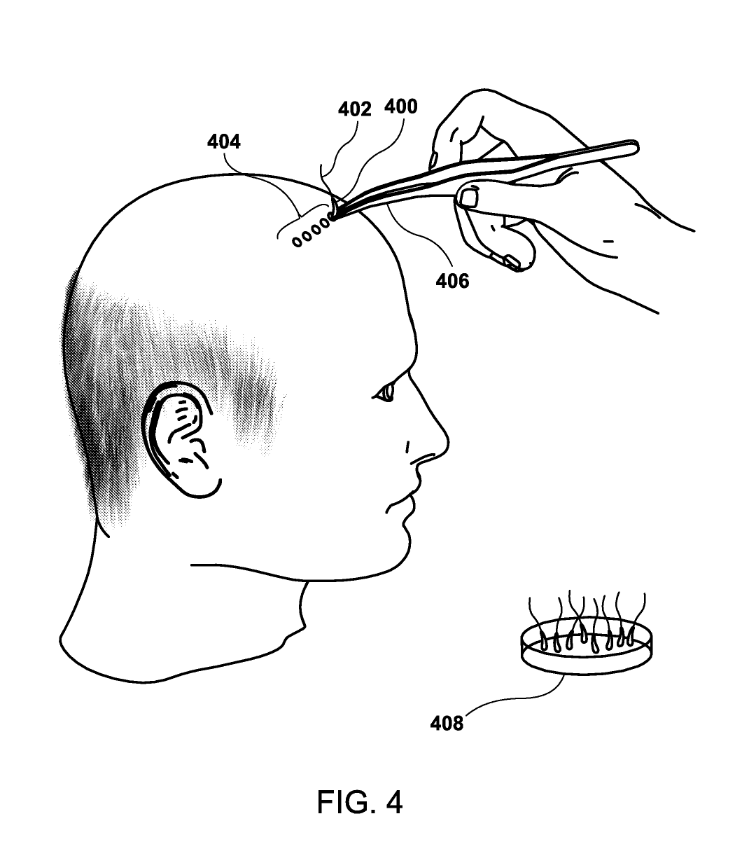

FIG. 3B depicts a tissue sample that includes a hair follicle (306) obtained in FIG. 3A, FIG. 4 generally refers to a subject in need or desiring a follicular transplant receiving a follicular transplant (400). The subject in FIG. 4 is preferably the same subject in FIG. 3A, but that need not be the case. At least one hair follicle implant (402) is implanted into at least one implantation locus or group of implantation loci (404) preferably made using surgical techniques and tools, but that need not be the case, using at least one implantation tool (406). The hair follicle implant is taken from a culture of at least one 3D printed cultured hair follicles or collection of cells of the present invention (408).

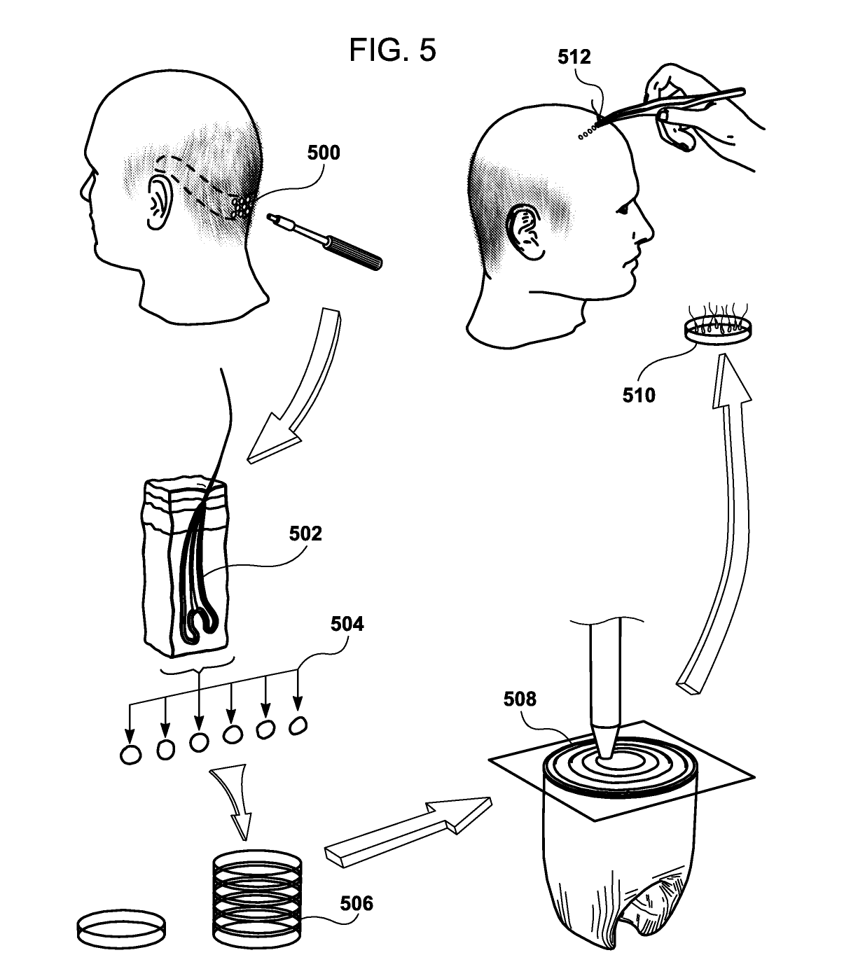

FIG. 5 generally refers to a collage of FIG. 1, FIG. 2, FIG. 3A, FIG. 3B and FIG. 4 depicting one preferred aspect of the present invention. The method begins with extraction of at least one hair follicle with a follicle extraction tool preferably from a follicle extraction zone of a subject (500). The at least one hair follicle (502) extracted is used to obtain at least one cell (504), at least one type of cell, or a combination thereof, capable of forming a new functional hair follicle when used in the methods of the present invention. These cells are provided in an appropriate culture medium (506) and are 3D printed into a collection of cells that can form at least one hair follicle (508). The printed collection of cells are optionally cultured, grown and prepared for implantation into a subject, preferably the same subject from whom the cells were obtained from to preferably obtain at least one functional hair follicle (510). The 3D printed collection of cells that can form at least one hair follicle, or functional hair follicles made therefrom, are then inserted into a subject in need or desiring a follicular transplant (512).

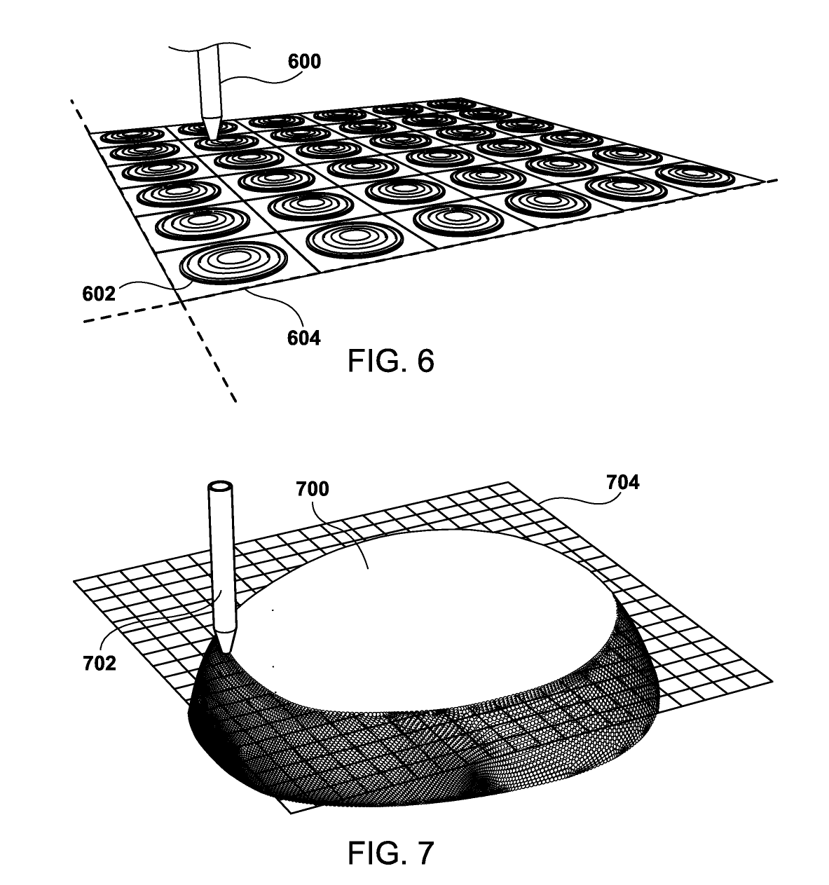

FIG. 6 generally refers to a 3D printer nozzle (600) printing at least one printed cell layer (602) onto a growth medium, well, plate, or a combination thereof, such as in the form of a matrix. The concurrent 3D printing at least one layer of cells, at least one hair follicle, multiple hair follicles, or a combination thereof, is performed on an X, Y, and Z plane (604) to build at least one portion of at least one functional hair follicle.

FIG. 7 generally refers to 3D printing of a patch of follicle-rich implantable structure (700) in a shape that generally matches the topology of the area in general on a subject that is to receive the 3D printed patch of follicle-rich skin. The printer nozzle (702) can be utilized to print and build an appropriate three-dimensional structure of the 3D printed patch of follicle-rich skin, by 3D printing onto a cross-sectional plan (704) guided computer assisted equipment.

### I Method of Making a Collection of Cells Capable of Forming a Functional Hair Follicle

A first aspect of the present invention is a method of making at least one three dimensional collection of cells capable of forming a functional hair follicle, including a) providing at least one three dimensional printable formulation including cells capable of forming a functional hair follicle; and b) three dimensional printing with the at least one three dimensional printable formulation, at least one three dimensional collection of cells capable of forming at least one functional hair follicle.

As to the printable formulation in the present invention, the at least one three dimensional printable formulation includes materials, including but not limited to: cell suspensions, biologically active materials, pluripotent stem cells, differentiated cells, growth factors, cell differentiation signaling molecules, cell stabilizing factors, fibrin, fibronectin, laminin, extra cellular matrix molecules (ECM), or a combination thereof, being either a homogenous or heterogeneous mixture of cells, which are capable of forming a functional hair follicle under appropriate conditions.

The printable formulation of the present invention can also include printable formulations permitting three-dimensional printability permitting adhesion, orientation, or a combination thereof including, but not limited to: fibrin, fibronectin, laminin, collagen gels, viscosity modifying agents including surfactants and humectants, alginate solutions, calcium chloride solutions, or a combination thereof. The at least one three dimensional printable formulation of the present invention is composed such that cell adhesion, cell fusion, and appropriate three dimensional orientation of multicellular aggregates allow for the formation of said three dimensional printable formulation into a desired shape, including a hair follicle.

Regarding the printable formulation in the present invention, three-dimensional printing with said at least one three dimensional printable formulation is accomplished by methods and techniques including, but not limited to: printing onto a predefined structure of correct geometric characteristics resembling that of the needed biological structure, including a hair follicle; printing using computer aided template to guide geometric, architectural, and relative positioning characteristics resembling that of the needed biological structure, including a hair follicle, printing onto a scaffold resembling the characteristics of the biological tissue, organ, or structure of interest, including a hair follicle.

As to three-dimensional printing in general, preferably using the at least one three dimensional printable formulation, is accomplished by methods and techniques including, but not limited to: printing a three dimensional aggregate of cells with the printable formulation outside of a scaffold, gel, or any such pre-designed molding. The printing of the three dimensional printable formulation is such that the geometry, architecture, and relative physical orientation of cells aggregate to one another permitting formation of native tissue structures, and viable functionality permitting implantation. The three dimensional printing with at least one three dimensional printable formulation is accomplished in ways that are generally known in the art, for example, as reported by Organovo-Invetech, San Diego Calif., and Neatco, Carlisle Canada. The appropriate equipment and methods for three dimensional printing are known generally in the art, which are incorporated herein by reference in their totality, and in particularly for the purpose of three dimensional printing of tissues (Karoly Jakab, Cyrille Norotte, Francoise Marga, KeithMurphy, Gordana Vunjak-Novakovic, and Gabor Forgacs. Tissue engineering by self-assembly and bio-printing of living cells. Biofabrication 2 (2010) 022001; and Chirag Khatiwala, Richard Law, Benjamin Shepherd, Scott Dorfman, and Marie Csete. 3D Cell Bioprinting For Regenerative Medicine Research And Therapies. Gene Therapy and Regulation Vol. 7, No. 1 (2012)).

With regards to three-dimensional printing, the equipment and methods can be those generally known in the art, which are incorporated herein by reference in their totality, and in particularly for the purpose of three-dimensional printing of tissues, can be used to achieve in general at least one aspect of the present invention. Preferably, the three dimensional printing can be performed by methods and equipment including, but not limited to: 3D-Bioprinter (Regenovo Biotechnology Co., Ltd), TiVido BioDevices, n3D Biocsicnes, Rainbow BioSciences, EnvisionTec pritners (Ultra® 3SP™ Family, and Prefactory® Family), GeSIM, Organovo: NovoGen MMX, or a combination thereof.

In one aspect of the present invention, the at least one functional hair follicle is capable of viable transplantation into a subject.

As to the at least one functional hair follicle of the present invention, the at least one functional hair follicle can be transplanted into a subject in need, including a human, by follicular unit transplantation. The present invention allows for an essentially unlimited access of viable follicles without the need for follicular extraction using the follicular extraction method, or any such methods. The present invention allows for at least one viable functioning hair follicle to be transplanted into a subject, including a human, without provoking any “anti-self” cellular and molecular mechanisms from the body of the recipient.

In another aspect of the present invention, the method is performed ex vivo.

Regarding the method of the present invention, the method can be performed in vitro, such as in or on petri dishes with solid growth media, in micro-wells, using bioreactors, using incubators, or a combination thereof; on or inside of hydrogels, bio-gels, synthetic scaffolds, biological scaffolds, or a combination thereof. The present invention allows for culturing, replication, growth, or a combination thereof, of hair follicles ex vivo with methods established in the art and combined, at least in part or entirely, with the methods described in the present invention to obtain at least one viable, transplantable hair follicle capable of anagenic growth once implanted into a subject.

As to the three dimensional collection of cells of the present invention, the three dimensional collection of cells including, but not limited to: an entire hair follicle, a portion of a hair follicle, a substantial portion of a hair follicle, at least one portion of a hair follicle, biological materials capable of growing at least in part at least one terminal hair, at least one medulla, at least one cortex, at least one cuticle, at least one inner root sheath, at least one outer root sheath, or a combination thereof, combined together by the methods mentioned herein, and by other methods that can be developed to achieve a similar purpose, are utilized to generate an unlimited amount of hair follicle cells, or any precursors capable of becoming hair follicle cells, ex vivo, which are implantable and accepted by the subject recipient.

In a further aspect of the present invention, the three dimensional collection of cells includes an entire hair follicle, a portion of a hair follicle, a substantial portion of a hair follicle, at least one portion of a hair follicle, at least one dermal papilla, at least one hair matrix cell, at least one bulge region, biological materials capable of growing at least in part at least one terminal hair, at least one medulla, at least one cortex, at least one cuticle, at least one inner root sheath, at least one outer root sheath, or a combination thereof.

The referred to three dimensional collection of cells can be present in proportions and distributions including: the collection of cells in their entirety, as a portion of, as a substantial portion of, in at least one portion, or a combination thereof, within an ink cartridge of a 3D printer capable of biological printing, being present on or as a scaffold, being present on, in, or within a gel, being present on, in, or within a hydrogel, being present as part of the printing medium, or a combination thereof.

In an additional aspect of the present invention, the three-dimensional printing prints layers of cells in a configuration to produce a viable hair follicle.

As to the configuration of the three dimensional printing of the present invention, the printing configuration allowing for production of a viable hair follicle can be achieved by methods including, but not limited to: computer assisted designs, pre-designed physical structures to be printed onto, a scaffold to print onto, a physical structure containing a negative space representing the final structure, or a combination thereof, to produce a viable hair follicle.

In another aspect of the present invention, the three dimensional printable formulations includes cells, stem cells, synthetic biological molecules, or a combination there of.

Referring to cells including the printable formulations include, such include but are not limited to: at least one central layer cell of the follicle that forms the hair shaft, at least one outer layer cell of the follicle that forms the root sheath, at least one cell capable of forming the inner root sheath, at least one papilla, at least one mesodermal fibroblast capable of forming at least one dermal papilla, at least one keratinocyte, or a combination thereof.

As to the stem cells in the present invention, the stem cells comprising the printable formulations include, but are not limited to: at least one mesodermal stem cell, at least one fibroblast, at least one ectodermal stem cells, or a combination thereof.

Regarding the synthetic biological molecules in the present invention, the synthetic biological molecules comprising the printable formulations include, but are not limited to: at least one growth factor, at least one cell differentiation factor, at least one pre-dermal papilla signaling molecule, at least one fibroblast signaling molecule, at least one mesoderm signaling molecule, at least on anagenic signal, at least on catagenic signal, at least one telogenic signal, at least one exogenic signal, at least one keratinocyte differentiating signal, or a combination thereof.

In a further aspect of the present invention, the printable formulation is provided in a bio-printer cartridge.

Generally referring to the bio-printer cartridge, such devices and articles of manufacture include at least one orifice, which can be conducive to certain features including, but no limited to: outward flow, minimum shear pressure on cells, minimum friction of the printable formulation, prevention of clumping, prevention of bottleneck, prevention of clogging, or a combination thereof.

Preferably, the bio-printer cartridge includes: at least one orifice, at least one nozzle, no nozzle, or a combination thereof. Configurations of such bio-printer cartridges are known and available in the art.

Regarding the bio-printer in the present invention, the bio-printer can also include at least one dispensing head, with at least one printable formulation contained in at least one dispensing head. The at least one printable formulation can be prefilled, replenished, replaced, or a combination thereof, into the at least one dispensing head of the bio-printer. Configurations of such bio-printer cartridges are known and available in the art.

In an additional aspect of the present invention, the printable formulation is provided in a bio-printer.

The printable formulation can be provided in a bio-printer by methods including, but not limited to: direct injection, prefilled dispensing heads, replaceable dispensing heads, refillable dispensing heads, prefilled cartridges, replaceable cartridges, refillable cartridges, or a combination thereof.

In another aspect of the present invention, the subject is an organism having hair follicle structures, a mammal, a human, non-human primates, a veterinary animal, an agricultural animal, a wildlife animal, a test animal, a laboratory animal, a mouse, a rat, a guinea pig, a dog, a cat, a pig, a ferret, or a combination thereof.

Preferably, the subject can derive at least one benefit from receiving the methods described herein. The subject can benefit from the present invention due to necessity, due to want, or a combination thereof.

In a further aspect of the present invention, the at least one three dimensional collection of cells includes at least one scaffold.

The at least one scaffold can include materials including, but not limited to: no scaffold, at least in part at least one scaffold, at least one scaffold, a synthetic scaffold, a biological scaffold, a synthetic polymer, a biological polymer, poly-lactic-co-glycolic acid (PLGA), alginate solutions, calcium chloride solutions, fibrin, fibronectin, collagen, elastacin, laminin, extra cellular matrix (ECM), chitosan, glycosaminoglycans (GAGs), heparin, heparan sulfate, hydroxyapatite, chitin, chondroitin sulfate and hyaluronic acid, proteoglycan decorin, glycoproteins, biglycan, entactin, or a combination thereof.

In addition, the at least one three dimensional collection of cells can include: no gel, at least in part at least one gel, at least one gel. As to the gel in the present invention, the at least one gel can include materials such as but not limited to at least one hydrogel. Preferably, the at least one hydrogel can include a therapeutically safe and effective amount of at least one biodegradable hydrogel including, but not limited to: at least one polymer, at least one hydrophilic polymer, or a combination thereof. Preferably, the at least one polymer can include, but is not limited to: polysaccharides, beta-linked acetylated mannan, Xylitol, glyceryl acrylate, glyceryl polyacrylate, chitosan, or a combination thereof; the at least one hydrophilic polymer can include, but is not limited to: Poly(N-isopropylacrylamide) (PNIPAM) and Polyacrylamide (PAM), Poly(2-oxazoline) and Polyethylenimine (PEI), Poly(acrylic acid), Polymethacrylate and Other Acrylic Polymers, Poly(ethylene glycol) and Poly(ethylene oxide), Poly(vinyl alcohol) (PVA) and Copolymers, Poly(vinylpyrrolidinone) (PVP) and Copolymers, Polyelectrolytes, Cucurbit[n]uril Hydrate, or a combination thereof.

In the present invention, the no gel, at least in part at least one gel, at least one gel can include material such as, but not limited to: at least in part at least one biological gel, at least in part at least one synthetic gel. The at least in part at least one biological gel can include materials such as but not limited to: collagen cross-linkages, collagen fibrillar gel, fibrin gels, collagen gels, elastic polymers, non-elastic polymers, or a combination thereof.

In another aspect of the present invention, the at least one three dimensional collection of cells comprises at least one cell-free or substantially cell-free space.

Preferably, the at least one cell-free or substantially cell-free space can occupy negative space as a placeholder during 3D printing. The at least on cell-free or substantially cell-free space can be comprised of materials including, but not limited to: any scaffold including a synthetic scaffold, a biological scaffold, a synthetic polymer, a biological polymer, poly-lactic-co-glycolic acid (PLGA), alginate solutions, calcium chloride solutions, fibrin, fibronectin, collagen, elastacin, laminin, extra cellular matrix (ECM), chitosan, glycosaminoglycans (GAGs), heparin, heparan sulfate, hydroxyapatite, chitin, chondroitin sulfate and hyaluronic acid, proteoglycan decorin, glycoproteins, biglycan, entactin, or a combination thereof; any hydrogel including at least one hydrogel can include a therapeutically safe and effective amount of at least one biodegradable hydrogel including, but not limited to: at least one polymer, at least one hydrophilic polymer, or a combination thereof. Preferably, the at least one polymer can include, but is not limited to: polysaccharides, beta-linked acetylated mannan, Xylitol, glyceryl acrylate, glyceryl polyacrylate, chitosan, or a combination thereof; the at least one hydrophilic polymer can include, but is not limited to: Poly(N-isopropylacrylamide) (PNIPAM) and Polyacrylamide (PAM), Poly(2-oxazoline) and Polyethylenimine (PEI), Poly(acrylic acid), Polymethacrylate and Other Acrylic Polymers, Poly(ethylene glycol) and Poly(ethylene oxide), Poly(vinyl alcohol) (PVA) and Copolymers, Poly(vinylpyrrolidinone) (PVP) and Copolymers, Polyelectrolytes, Cucurbit[n]uril Hydrate; any gel including at least in part at least one biological gel, at least in part at least one synthetic gel. The at least in part at least one biological gel can be comprised of materials including, but not limited to: collagen cross-linkages, collagen fibrillar gel, fibrin gels, collagen gels, elastic polymers, non-elastic polymers, or a combination thereof.

### II A Collection of Cells Capable of Forming a Functional Hair Follicle

A second aspect of the present invention is a product produced by the method of the present invention described in I above and herein.

As to the product produced by the method of the present invention describe in I and herein, the produced product includes, but is not limited to: at least in part at least one hair follicle, a portion of a hair follicle, a substantial portion of a hair follicle, at least one portion of a hair follicle, at least one entire hair follicle, at least in part at least one terminal hair, at least one medulla, at least one cortex, at least one cuticle, at least one inner root sheath, at least one outer root sheath, at least in part at least one bulge, or a combination thereof, combined together by the methods mentioned herein, and by other methods that can be developed to achieve a similar purpose, are utilized to generate an unlimited amount of hair follicle cells, at least one dermal papilla, or any precursors capable of becoming hair follicle cells, or a combination thereof.

### III A Method of Making a Functional Hair Follicle

A third aspect of the present invention is a method of making at least one functional hair follicle, including: a) providing a product produced by the method of the present invention described in I above and herein, b) culturing the product produced by the method of the present invention described in I above and herein under culturing conditions to produce at least one functional hair follicle

Referring to the providing a product produced by the method of the present invention described in I above and herein, the product produced includes, but is not limited to: at least one three dimensional collection of cells, at least in part at least one hair follicle, at least one portion of at least one hair follicle, a substantial portion of at least one hair follicle, at least one entire hair follicle, at least in part at least one terminal hair, at least one portion of at least one terminal hair, a substantial portion of a terminal hair, at least one terminal hair, at least one portion of at least one medulla, a substantial portion of at least one medulla, at least one medulla, at least one portion of at least one cortex, a substantial portion of at least one cortex, at least one cortex, at least one portion of at least one cuticle, a substantial portion of at least one cuticle, at least one cuticle, at least one cuticle, at least one portion of at least one inner root sheath, a substantial portion of at least one inner root sheath, at least one inner root sheath, at least one portion of at least one outer root sheath, a substantial portion of at least one outer root sheath, at least one outer root sheath, at least one portion of at least one bulge region, a substantial portion of at least one bulge region, at least one bulge region, at least one portion of at least one dermal papilla, a substantial portion of at least one dermal papilla, at least one dermal papilla, at least one portion of at least one hair matrix cell, a substantial portion of at least one hair matrix cell, at least one hair matrix cell, or a combination thereof.

With regards to the culturing the product produced by the method of the present invention described in I above and herein under culturing conditions to produce at least one functional hair follicle, the culturing conditions include, but are not limited to: culturing in bioreactors, culturing in incubators, culturing at the proper physiological conditions conducive to producing the product of the present invention, providing at least in part at least one molecule, providing at least in part at least one molecular precursor, providing at least in part at least one growth factor, or a combination thereof; providing the proper temperature for culturing at least one cell, at least one group of cells, at least in part at least one tissue, with a minimum temperature of 2 degrees Celsius and a maximum degree of 104 degrees Celsius. The methods for culturing at least one functional hair follicle, culturing stem cells, or a combination thereof, are known generally in the art and are incorporated by reference herein in their totality, and in particular for culturing the product produced by the method of the present invention described in I above and herein (Weterings, P. J. J. M., Vermorken, A. J. M. and Bloemendal, H. (1981), A method for culturing human hair follicle cells. British Journal of Dermatology, Vol. 104: 1-5; and Messenger, A. G. (1984), The culture of dermal papilla cells from human hair follicles. British Journal of Dermatology. Vol. 110: 685-689), each of which is incorporated by reference in their entirety herein.

In another aspect of the present invention, the culturing conditions include growth factors, growth complexes, stem cells, synthetic materials, biological materials, anagenic compounds, or a combination thereof

With respect to the culturing conditions include growth factors, growth complexes, stem cells, synthetic materials, biological materials, anagenic compounds, or a combination thereof, the culturing conditions include conditions that are known generally in the art and are incorporated by reference herein in their totality, and in particular for culturing the product produced by the method of the present invention described in I above and herein (Philpott, M. P.; Sanders, D. A.; Kealey, T. Journal of Investigative Dermatology. 1994, Vol. 102 (6), p 857-861. 5p.; C. A. B. Jahoda*, K. A. Horne & R. F. Oliver. Induction of hair growth by implantation of cultured dermal papilla cells. Nature. 1984, Vol. 311, 560-562; Weterings, P. J. J. M., Vermorken, A. J. M. and Bloemendal, H. (1981), A method for culturing human hair follicle cells. British Journal of Dermatology, Vol. 104: 1-5; Leirós G. J., Attorresi A. I., Balañá M. E. Hair follicle stem cell differentiation is inhibited through cross-talk between Wnt/β-catenin and androgen signaling in dermal papilla cells from patients with androgenetic alopecia. Br. J. Dermatol. 2012; 166(5):1035-42; Oh J. H., Mohebi P., Farkas D. L., Tajbakhsh J. Towards expansion of human hair follicle stem cells in vitro. Cell Prolif. 2011; 44(3):244-53; and Messenger, A. G. (1984), The culture of dermal papilla cells from human hair follicles. British Journal of Dermatology. Vol. 110: 685-689), each of which is incorporated by reference in their entirety herein.

In a further aspect of the present invention, the at least one functional hair follicle is capable of viable transplant into a subject.

As to the at least one functional hair follicle is capable of viable transplant into a subject, the at least one functional hair follicle, and the transplantation methods are generally known in the art, and are incorporated by reference herein in their totality, and in particular for the transplantation of at least one functional hair follicle (Yasuyuki Amoh, Lingna Li, Raul Campillo, Katsumasa Kawahara, Kensei Katsuoka, Sheldon Penman, and Robert M. Hoffman Implanted hair follicle stem cells form Schwann cells that support repair of severed peripheral nerves. Vol. 102 (49). Yasuyuki Amoh, 17734-17738, doi: 10.1073/pnas.0508440102; Rassman, W. R., Bernstein, R. M., McClellan, R., Jones, R., Warton, E. and Uyttendaele, H. (2002), Follicular Unit Extraction: Minimally Invasive Surgery for Hair Transplantation. Dermatologic Surgery, 28: 720-728. doi: 10.1046/j.1524-4725.2002.01320.x), each of which is incorporated by reference in their entirety herein.

### IV A Functional Hair Follicle

A fourth aspect of the present invention is a product produced by the method of the present invention described in III above and herein.

As to the product produced by the method of the present invention described in III above and herein, the functional hair follicle is produced by methods described above and herein, methods generally known in the art, or a combination thereof, and are incorporated by references herein in their totality, and in particular for producing a functional hair follicle (Ariane Rochat, Koji Kobayashi, Yann Barrandon. Location of stem cells of human hair follicles by clonal analysis. Cell. 1994, Vol. 76 (6): 1063-1073; Philpott, M. P.; Sanders, D. A.; Kealey, T. Journal of Investigative Dermatology. 1994, Vol. 102 (6), p 857-861. 5p.; C. A. B. Jahoda*, K. A. Horne & R. F. Oliver. Induction of hair growth by implantation of cultured dermal papilla cells. Nature. 1984, Vol. 311, 560-562; Weterings, P. J. J. M., Vermorken, A. J. M. and Bloemendal, H. (1981), A method for culturing human hair follicle cells. British Journal of Dermatology, Vol. 104: 1-5; Leirós G. J., Attorresi A. I., Balañá M. E. Hair follicle stem cell differentiation is inhibited through cross-talk between Wnt/β-catenin and androgen signaling in dermal papilla cells from patients with androgenetic alopecia. Br. J. Dermatol. 2012; 166(5):1035-42; Oh J. H., Mohebi P., Farkas D. L., Tajbakhsh J. Towards expansion of human hair follicle stem cells in vitro. Cell Prolif. 2011; 44(3):244-53; Messenger, A. G. (1984), The culture of dermal papilla cells from human hair follicles. British Journal of Dermatology. Vol. 110: 685-689; V. A. Randall, M. J. Thornton and A. G., Messenger Cultured dermal papilla cells from androgen-dependent human hair follicles (e.g. beard) contain more androgen receptors than those from non-balding areas of scalp. Journal of Endocrinology. 1992, Vol. 133, 141-147; and Hong Yu, Dong Fang, Suresh M. Kumar, Ling Li, Thiennga K. Nguyen, Geza Acs, Meenhard Herlyn, Xiaowei Xu. Isolation of a Novel Population of Multipotent Adult Stem Cells from Human Hair Follicles. American J. of Pathology. 2006, Vol. 168 (6): 1879-1888), each of which is incorporated by reference in their entirety herein.

### V Method of Hair Growth Using a Collection of Cells Capable of Forming a Functional Hair Follicle

A fifth aspect of the present invention is a method of hair growth in a subject, including: a) providing a subject in need or desiring hair growth; b) providing the product produced by the method of the present invention described in II above and herein; and c) transplanting the product of the present invention described in II above into said subject; wherein the product produced by the method of the present invention described in II above and herein.

In regards to providing a subject in need or desiring hair growth, the subject includes, but is not limited to: an organism having hair follicle structures, a mammal, a human, non-human primates, a veterinary animal, an agricultural animal, a wildlife animal, a test animal, a laboratory animal, a mouse, a rat, a guinea pig, a dog, a cat, a pig, a ferret, or a combination thereof. Preferably, the subject can derive at least one benefit from receiving the methods described herein. The subject can benefit from the present invention due to necessity, due to want, or a combination thereof.

As to the providing the product produced by the method of the present invention described in II above and herein, the product produced is accomplished by the method of I, II, III, or a combination thereof, producing at least in part a collection of cells capable of forming at least in part at least one functional hair follicle capable of hair growth. The product produced by the method of the present invention described in II above and herein, can be used to produce at least in part a collection of cells capable of forming at least in part at least one functional hair follicle capable of hair growth to grow at least one viable hair, group of hairs, or a combination thereof, ex vivo; can be used to produce at least in part a collection of cells capable of forming at least in part at least one functional hair follicle capable of being implanted into said subject in need or desiring hair growth in vivo, being able to undergo anagenic hair growth of the implanted hair follicle into said subject after transplantation.

Referring to transplanting the product of the present invention described in II above into said subject; wherein the product produced by the method of the present invention described in II above and herein, the product can be transplanted into said subject to produce viable hair growth by methods generally known in the art (Yasuyuki Amoh, Lingna Li, Raul Campillo, Katsumasa Kawahara, Kensei Katsuoka, Sheldon Penman, and Robert M. Hoffman. Implanted hair follicle stem cells form Schwann cells that support repair of severed peripheral nerves. Vol. 102 (49). Yasuyuki Amoh, 17734-17738, doi: 10.1073/pnas.0508440102; Rassman, W. R., Bernstein, R. M., McClellan, R., Jones, R., Worton, E. and Uyttendaele, H. (2002), Follicular Unit Extraction: Minimally Invasive Surgery for Hair Transplantation. Dermatologic Surgery, 28: 720-728. doi: 10.1046/j.1524-4725.2002.01320.x), each of which is incorporated by reference in their entirety herein.

### VI Method of Hair Growing Using Functional Hair Follicles

A sixth aspect of the present invention is a method of hair growth in a subject, including: a) providing a subject in need or desiring hair growth; b) providing the product produced by the method of the present invention described in IV above and herein; c) transplanting the product produced by the method of the present invention described in IV above and herein into said subject; wherein the product produced by the method of the present invention described in IV above and herein produces hair growth in said subject.

As to the providing a subject in need or desiring hair growth, the subject includes, but is not limited to: an organism having hair follicle structures, a mammal, a human, non-human primates, a veterinary animal, an agricultural animal, a wildlife animal, a test animal, a laboratory animal, a mouse, a rat, a guinea pig, a dog, a cat, a pig, a ferret, or a combination thereof. Preferably, the subject can derive at least one benefit from receiving the methods described herein. The subject can benefit from the present invention due to necessity, due to want, or a combination thereof.

With regard to providing the product produced by the method of the present invention described in IV above and herein, the product produced is accomplished by the method of I, II, III, or a combination thereof, producing at least in part at least one functional hair follicle capable of hair growth. The product produced by the method of the present invention described in IV above and herein, can be used to produce at least in part at least one functional hair follicle capable of hair growth to grow at least one viable hair, group of hairs, or a combination thereof, ex vivo; can be used to produce at least in part at least one functional hair follicle capable of hair growth to grow at least one viable hair, group of hairs, or a combination thereof, in vivo, being able to undergo anagenic hair growth of the implanted hair follicle into said subject after transplantation.

Referring to transplanting the product produced by the method of the present invention described in IV above and herein into said subject; wherein the product produced by the method of the present invention described in IV above and herein produces hair growth in said subject, the product can be transplanted into said subject to produce viable hair growth by methods generally known in the art (Yasuyuki Amoh, Lingna Li, Raul Campillo, Katsumasa Kawahara, Kensei Katsuoka, Sheldon Penman, and Robert M. Hoffman Implanted hair follicle stem cells form Schwann cells that support repair of severed peripheral nerves. Vol. 102 (49). Yasuyuki Amoh, 17734-17738, doi: 10.1073/pnas.0508440102; Rassman, W. R., Bernstein, R. M., McClellan, R., Jones, R., Worton, E. and Uyttendaele, H. (2002), Follicular Unit Extraction: Minimally Invasive Surgery for Hair Transplantation. Dermatologic Surgery, 28: 720-728. doi: 10.1046/j.1524-4725.2002.01320.x), each of which is incorporated by reference in their entirety herein.

### EXAMPLES

### Example 1

### Production of Hair Follicle Structures

This example establishes methods and compositions for the making of biologically active hair follicles for insertion into a subject, such as a human, for hair restoration, utilizing cell based printing, such as 3D printing.

### Obtaining and Culturing Cells for 3D Printing

Cells and keratins are multiplied in the lab with existing techniques. Then they can be used to form, create, and grow ready to be inserted hair follicles, or can be placed in the printer cartridges to 3D print such ready to be inserted hair follicles. These cells can be treated with necessary growth factors, complexes, and chemicals, or other appropriate materials, before printing.

US Published Patent Application No. 2010/017683 to Barrowes et al. provide a summery of the use of neonatal foreskin or plucked hairs for fibroblasts and keratinocytes, which can be used in the present invention. Certain sections of the following are from or adapted from that document.

The tissue engineered skin may be prepared by any suitable method known to one skilled in the art. For example, human neonatal foreskin tissue can be used as a source of human dermal fibroblasts that are multiplied in culture and seeded onto a scaffold such as collagen gel to provide a tissue engineered dermal layer. Epidermal keratinocytes can be obtained from the same neonatal tissue or, alternatively, obtained from plucked hair follicles. A tissue engineered epidermal layer can be produced from plucked hair follicles as disclosed in U.S. Pat. Nos. 6,730,513, 6,673,603, 6,548,058, 5,968,546, and references cited therein, the teachings of which are incorporated by reference herein. The tissue engineered dermal layer and tissue engineered epidermal layer can be separately prepared and then assembled into tissue engineered skin with hair follicle progenitor cells dispersed therein, suitably sandwiched between the two assembled layers. The tissue engineered dermal and epidermal layers can be prepared without the use of a scaffold, for example by the method described by Pouliot, et al. in Transplantation, 2002 Jun. 15; 73(11):1751-7, and references cited therein, the teachings of which are incorporated by reference herein. See, US Published Patent Application No. 2010/017683 to Barrowes et al. at paragraph [0008].

US Published Patent Application No. 2010/017683 and U.S. Pat. No. 7,597,885 to Barrowes et al. provide a summery of the use of specific combination of cultured cells for follicle neogenesis, which can be used in the present invention. Certain sections of the following is from or adapted from U.S. Pat. No. 7,597,885.

In yet another aspect, the present invention is a specific combination of cultured cells that provide reliable and reproducible initiation of follicle neogenesis. The implantation of cultured dermal papilla (also known as follicular papilla) cells alone gives unpredictable, non-reproducible results. It has been found that keratinocytes, suitably keratinocytes obtained from neonatal skin (e.g. infant foreskin tissue), also must be implanted in combination with the dermal papilla cells. Other types of cells optionally also can be combined with the dermal papilla/keratinocyte combination to improve the success rate of follicle neogenesis. Optional additional cells may be selected from stem cell populations that are known to exist in human embryo, fetal or infant scalp skin, infant foreskin, umbilical cord blood, adult bone marrow, muscle, adipose tissue, and skin. See, U.S. Pat. No. 7,597,885 to Barrowes et al. at column 2, lines 39 through 53.

Making 3D Printing Material Including Cells and Printing Using these Materials

Procedures, compositions, methods and devices for the 3D printing of cells, including printing for the production of tissues and organs, have been reported in the technical, patent and general media literature, and can be used in the present invention, along with later developed related technologies. Some representative patent, technical and media documents are US Published Patent Application No. 2012/0224755 to Wu, U.S. Pat. No. 7,087,200 to Taboas et al, U.S. Pat. No. 8,260,589 to Kumar, Mironove et al. “Organ Printing: Computer-aided Jet-based 3D tissue engineering,” organprint.missouri.edu/www/fibr-pub/mironov03-157.pdf (public availability date not available, copy obtained Jun. 27, 2013), Marga et al. “Toward engineering functional organ modules by additive manufacturing” Biofacation, 4:1012 (2012), Khatiwala et al. “3D cell bioprinting for regenerative medicine research and therapies” Gene Therapy and Regulation 7(1):1-19 (2012), www.Organonovo.com materials relating to 3D cell printing (Jul. 5, 2012), “3D bioprinting with autologous cells could prevent organ transplant rejections, says Organovo Exec. VP” May 10, 2013, medtechinsider.com/archives/3140, Takebe et al, “Self-organization of human hepatic organoid by recapitulating organogenesis in vitro” Transplantation Proceedings 44:1018-1020 (2012), and Mannoor et al. “3D printed bionic ears” Nano Lett. 13:2634-2639 (2013), the technologies therein are applicable for use in the present invention and are incorporated by reference herein.

### Printing a Hair Follicle Using 3D Printing

Using 3D printing, an entire terminal hair, or parts of, or biological material that is capable of growing a terminal hair, or parts of, is performed. Parts of terminal hair include but are not limited to: medulla, cortex, cuticle, inner root sheath, outer root sheath etc.

A 3 dimensional printer prints objects with respect to the mathematical x, y, and z axis. Instead of printing a design on a flat sheet of paper, it can create tangible objects with shape and definition. These objects may be solid and may have mass. This is accomplished, for example, by printing multiple layers in 1 axis with respect to the other 2 axes.

A 3 dimensional printer has the capability of printing viable and functional human organs. Rather than ink or plastics, multiple types of viable cells are placed into the printing cartridges. The printer places layers of the proper types of cells in the proper order to create the necessary architecture and structure of a viable tissue or organ. Proper 3 dimensional order of cell types is desirable for tissues and organs to carry out biological functions that are reliant on the relationships of cell types to interact properly with one another.

A hair follicle is constructed of cells and keratins in cylindrical layers. These layers and structures contain, and are not limited to: The inner most layer is the medulla. The medulla is surrounded by the cortex. The cortex is surrounded by the cuticle. The cuticle is surrounded by the inner root sheath. The inner root sheath is surrounded by the outer root sheath. The most deep to the skin or inferior part of a hair follicle contains the dermal papilla and the martical cells. The desirable layers described here can be created and printed with appropriate 3 dimensional structure to represent a viable hair follicle, with the desirable spatial relationships of specific cells and keratins.

### Culturing Printed Hair Follicles

These premade and ready to be inserted into a subject hair follicles can be treated with necessary growth factors, complexes, chemicals and other materials before insertion into a subject as described herein, and can be provided after insertion into a subject. The use of growth factors in such methods has been described herein. See, for example, US Published Patent Application No. 2005/0214344 and U.S. Pat. No. 7,597,885 to Barrowes et al.

### Example 2

### Protocol for Practicing Representative Methods of the Present Invention

This example provides a protocol for practicing various aspects of the present invention. This protocol is just one aspect of the present invention and is not intended to be limiting in nature.

1. Obtaining materials for premade follicles:
a. A skin biopsy with between about 10 and about 100 follicles is taken from the donor region of a subject using a punch biopsy tool or a scalpel. This would occur after the targeted area is locally anesthetized. Individual follicles are separated, preferably under a microscope. Components of hair follicles are multiplied and separated including cells, stem cells and keratins.
b. An alternative method is synthetic biological materials are created and multiplied that include hair follicle cells, stem cell and keratins.

2. Preparing “ink cartridges”
Various stem cells, cells and keratins are separated and placed in separate printer cartridges in a formulation that is compatible with the cells, optionally with growth factors and an appropriate cell growth medium, and printing media for 3D printing of cells. These materials may include but are not limited to: hair medulla, cortex, cuticle, inner root sheath, outer root sheath, dermal papilla, follicular matrix cells, etc. These printer cartridges with distinct biological follicular materials are loaded in a 3D printer, preferably a 3D printer compatible with printing cells.

3. Printing hair follicles.
Computer software capable of 3D printing is programmed to print a 3 dimensional hair follicle. Layer by layer (regarding z axis), proper biological components listed above are printed placing hair follicle cells on a surface in 2 dimensional orientation (x-y axis) cross sections. Since the printing is layer by layer (regarding z axis), the sum of layers printed on top of each other creates a 3 dimensional hair follicle with distinct cell and keratin locations regarding all x, y, and z axes. Follicular units may be printed in groups of 1, 2, 3 or multiple.

4. Collecting ex vivo made hair follicles.
This 3D printing of hair follicles is repeated anywhere between about 500 and about 10,000 times. These “ready to implant” lab-made hair follicles may be bathed in necessary growth factors and stored in a saline solution with proper tonicity, to prevent drying out.

5. Preparing the recipient region of a subject.
A hair restoration transplant surgeon or other appropriate health care professional will then anesthetize the recipient region of the scalp. Mini blades or needles that are between about 0.6 mm and about 2 mm thick are used to puncture holes in the skin in the recipient region in a density from between about 30 and about 70 punctures per cm². These punctures are made at a cosmetically beneficial density and angle where a hair is desired to grow.

6. Inserting the ex vivo made hair follicle
The physician or a technician will take a petri dish with the premade follicles. They will then use a forceps device to grab a follicular unit and insert it into the punctured skin hole in the recipient region, as would be done in traditional hair transplant technique.

All publications, including patent documents and scientific articles, referred to in this application and the bibliography and attachments are incorporated by reference in their entirety for all purposes to the same extent as if each individual publication were individually incorporated by reference.

All headings are for the convenience of the reader and should not be used to limit the meaning of the text that follows the heading, unless so specified.

1. A method of making at least one three dimensional collection of cells capable of forming a functional hair follicle, comprising:
a. providing at least one three dimensional printable formulation comprising cells capable of forming a functional hair follicle;
b. three dimensional printing with said at least one three dimensional printable formulation at least one three dimensional collection of cells capable of forming at least one functional hair follicle.

2. The method of claim 1,
wherein said at least one functional hair follicle is capable of viable transplantation into a subject.

3. The method of claim 1,
wherein said method is performed ex vivo.

4. The method of claim 1,
wherein said three dimensional collection of cells comprise an entire hair follicle, a portion of a hair follicle, a substantial portion of a hair follicle, at least one portion of a hair follicle, at least one dermal papilla, at least one hair matrix cell, at least one bulge region, biological materials capable of growing at least in part at least one terminal hair, at least one medulla, at least one cortex, at least one cuticle, at least one inner root sheath, at least one outer root sheath, or a combination thereof.

5. The method of claim 1,
wherein said three dimensional printing prints layers of cells in a configuration to produce a viable hair follicle.

6. The method of claim 1,
wherein said three dimensional printable formulations comprises cells, stem cells, synthetic biological molecules, or a combination there of.

7. The method of claim 1,
wherein said printable formulation is provided in a bio-printer cartridge.

8. The method of claim 1,
Wherein said printable formulation is provided in a bio-printer.

9. The method of claim 1,
wherein said subject comprises: an organism having hair follicle structures, a mammal, a human, non-human primates, a veterinary animal, an agricultural animal, a wildlife animal, a test animal, a laboratory animal, a mouse, a rat, a guinea pig, a dog, a cat, a pig, a ferret, or a combination thereof.

10. The method of claim 1,
wherein said at least one three dimensional collection of cells comprises at least one scaffold.

11. The method of claim 1,
wherein said at least one three dimensional collection of cells comprises at least one gel.

12. The method of claim 1,
wherein said at least one three dimensional collection of cells comprises at least one cell-free or substantially cell-free space.

13. (canceled)

14. A method of making at least one functional hair follicle, comprising:
a. providing the product of claim 1;
b. culturing said product of claim 1 under culturing conditions to produce at least one functional hair follicle

15. The method of claim 14,
wherein said culturing conditions comprise growth factors, growth complexes, stem cells, synthetic materials, biological materials, anagenic compounds, or a combination thereof

16. The method of claim 14,
wherein said at least one functional hair follicle is capable of viable transplant into a subject.

17. (canceled)

18. (canceled)

19. A method of hair growth in a subject, comprising:
a. providing a subject in need or desiring hair growth;
b. providing the product of claim 14;
c. transplanting said product of claim 14 into said subject;
wherein said product of claim 14 produces hair growth in said subject.
